SEQUENCE LISTING (50 sequences)

SEQ ID NO: 1 - RNA, 21 nt, Artificial
  synthetic oligonucleotide
gcuugaagucuuuaauuaann

SEQ ID NO: 2 - RNA, 21 nt, Artificial
  synthetic oligonucleotide
uuaauuaaagacuucaagcnn

SEQ ID NO: 3 - RNA, 21 nt, Artificial
  synthetic oligonucleotide
gcuugaagucuuuaauuaann

SEQ ID NO: 4 - RNA, 21 nt, Artificial
  synthetic oligonucleotide
uuaauuaaagacuucaagcnn

SEQ ID NO: 5 - RNA, 21 nt, Artificial
  synthetic oligonucleotide
gcnngaagucnnuaannaann

SEQ ID NO: 6 - RNA, 21 nt, Artificial
  synthetic oligonucleotide
uuaauuaaagacuucaagcnn

SEQ ID NO: 7 - DNA, 21 bp, Artificial
  synthetic oligonucleotide
gcttgaagtctttaattaagg

SEQ ID NO: 8 - RNA, 21 nt, Artificial
  synthetic oligonucleotide
uuaauuaaagacuucaagcnn

SEQ ID NO: 9 - RNA, 21 nt, Artificial
  synthetic oligonucleotides
gcuugaagucuuuaauuaann

SEQ ID NO: 10 - RNA, 21 nt, Artificial
  synthetic oligonucleotide
uuaauuaaagacuucaagcnn

SEQ ID NO: 11 - RNA, 21 nt, Artificial
  synthetic oligonucleotide
gcuugaagucuuuaauuaann

SEQ ID NO: 12 - RNA, 21 nt, Artificial
  synthetic oligonucleotide
nuaauuaaagacuucaagcnn

SEQ ID NO: 13 - RNA, 21 nt, Artificial
  synthetic oligonucleotides
gcuugaagucuuuaauuaann

SEQ ID NO: 14 - RNA, 21 nt, Artificial
  synthetic oligonucleotide
nnaannaaagacnncaagcnn

SEQ ID NO: 15 - RNA, 21 nt, Artificial
  synthetic oligonucleotide
gcuugaagucuuuaauuaann

SEQ ID NO: 16 - RNA, 21 nt, Artificial
  synthetic oligonucleotide
uuaauuaaagacuucaagcnn

SEQ ID NO: 17 - RNA, 21 nt, Artificial
  synthetic oligonucleotide
gcnngaagucnnuaannaann

SEQ ID NO: 18 - RNA, 21 nt, Artificial
  synthetic oligonucleotide
uuaauuaaagacuucaagcnn

SEQ ID NO: 19 - RNA, 21 nt, Artificial
  synthetic oligonucleotide
gcnngaagucnnuaannaann

SEQ ID NO: 20 - RNA, 21 nt, Artificial
  synthetic oligonucleotide
nuaauuaaagacuucaagcnn

SEQ ID NO: 21 - RNA, 21 nt, Artificial
  synthetic oligonucleotide
gcnngaagucnnuaannaann

SEQ ID NO: 22 - RNA, 21 nt, Artificial
  synthetic oligonucleotide
nnaannaaagacnncaagcnn

SEQ ID NO: 23 - RNA, 21 nt, Artificial
  synthetic oligonucleotide
gcnngaagucnnuaannaann

SEQ ID NO: 24 - RNA, 21 nt, Artificial
  synthetic oligonucleotide
uuaauuaaagacuucaagcnn

SEQ ID NO: 25 - DNA, 21 bp, Artificial
  synthetic oligonucleotide
gcttgaagtctttaattaagg

SEQ ID NO: 26 - RNA, 21 nt, Artificial
  synthetic oligonucleotide
uuaauuaaagacuucaagcnn

SEQ ID NO: 27 - DNA, 21 bp, Artificial
  synthetic oligonucleotide
gcttgaagtctttaattaagg

SEQ ID NO: 28 - DNA, 21 bp, Artificial
  synthetic oligonucleotide
nnaannaaagacnncaagcnn

SEQ ID NO: 29 - DNA, 21 bp, Artificial
  synthetic oligonucleotide
gcttgaagtctttaattaagg

SEQ ID NO: 30 - RNA, 21 nt, Artificial
  synthetic oligonucleotide
uuaauuaaagacuucaagcnn

SEQ ID NO: 31 - RNA, 21 nt, Artificial
  synthetic oligonucleotide
acacccuaugaauaugagunn

SEQ ID NO: 32 - RNA, 21 nt, Artificial
  synthetic oligonucleotide
acucauauucauagggugunn

SEQ ID NO: 33 - RNA, 21 nt, Artificial
  synthetic oligonucleotide
acacccuaugaauaugagunn

SEQ ID NO: 34 - RNA, 21 nt, Artificial
  synthetic oligonucleotide
acucauauucauagggugunn

SEQ ID NO: 35 - DNA, 21 bp, Artificial
  synthetic oligonucleotide
acaccctatgaatatgagttt

SEQ ID NO: 36 - RNA, 21 nt, Artificial
  synthetic oligonucleotide
acucauauucauagggugunn

SEQ ID NO: 37 - DNA, 21 bp, Artificial
  synthetic oligonucleotide
acaccctatgaatatgagttt

SEQ ID NO: 38 - RNA, 21 nt, Artificial
  synthetic oligonucleotide
acucauauucauagggugunn

SEQ ID NO: 39 - RNA, 21 nt, Artificial
  synthetic oligonucleotide
aagaacuugccuugauguann

SEQ ID NO: 40 - RNA, 21 nt, Artificial
  synthetic oligonucleotide
uacaucaaggcaaguucuunn

SEQ ID NO: 41 - RNA, 21 nt, Artificial
  synthetic oligonucleotide
aagaacuugccuugauguann

SEQ ID NO: 42 - RNA, 21 nt, Artificial
  synthetic oligonucleotide
uacaucaaggcaaguucuunn

SEQ ID NO: 43 - DNA, 21 bp, Artificial
  synthetic oligonucleotide
aagaacttgccttgatgtaca

SEQ ID NO: 44 - RNA, 21 nt, Artificial
  synthetic oligonucleotide
uacaucaaggcaaguucuunn

SEQ ID NO: 45 - DNA, 21 bp, Artificial
  synthetic oligonucleotide
aagaacttgccttgatgtaca

SEQ ID NO: 46 - RNA, 21 nt, Artificial
  synthetic oligonucleotide
uacaucaaggcaaguucuunn

SEQ ID NO: 47 - RNA, 21 nt, Artificial
  synthetic oligonucleotide
cccuacaccaagugauaaunn

SEQ ID NO: 48 - RNA, 21 nt, Artificial
  synthetic oligonucleotide
auuaucacuugguguagggnn

SEQ ID NO: 49 - RNA, 21 nt, Artificial
  synthetic oligonucleotide
cccuacaccaagugauaaunn

SEQ ID NO: 50 - RNA, 21 nt, Artificial
  synthetic oligonucleotide
auuaucacuugguguagggnn

(19) United States
(12) Patent Application Publication
(10) Pub. No.: US 2009/0298913 A1
(43) Pub. Date: Dec. 3, 2009

(54) SMALL INTERFERING OLIGONUCLEOTIDES COMPRISING ARABINOSE MODIFIED NUCLEOTIDES

(75) Inventors: Masad Damha, St. Hubert (CA); Nicolay Ferrari, Montreal (CA)

Correspondence Address: DAVID S. RESNICK, NIXON PEABODY LLP, 100 SUMMER STREET, BOSTON, MA, 02110-2131, US

(73) Assignee: TOPIGEN PHARMACEUTICALS INC., Montreal, QC (CA)

(21) Appl. No.: 12/091,318

(22) PCT Filed: Oct. 26, 2006
(86) PCT No.: PCT/CA06/01760

§ 371 Date: Oct. 15, 2008

Related U.S. Application Data
(60) Provisional application No. 60/730,876, filed on Oct. 28, 2005.
(60) Provisional application No. 60/741,544, filed on Dec. 2, 2005.

(51) Int. Cl. A61K 48/00; C07H 21/02; C07H 1/00

(52) U.S. Cl. 514/44 A; 536/24.5; 536/25.3

(57) ABSTRACT

Small interfering ribonucleic acid duplexes that inhibit gene expression containing at least one arabinose modified nucleotide are provided. Preferably, the duplexes contain ribonucleotides at least one arabinose modified nucleotide is 2′-deoxy-2′-fluoroarabinonucleotide (FANA) nucleotide.

### FIELD OF THE INVENTION

The invention relates generally to small interfering RNA duplexes (siRNA) containing at least one arabinose modified nucleotide, as well as small interfering 2′-deoxy-2′-fluoroarabinonucleic acid:RNA hybrids for the downregulation of gene expression.

### BACKGROUND OF THE INVENTION

Numerous strategies for silencing gene expression with nucleic acid-based molecules are under development [Stephenson, M. L. & Zamecnik, P. C. Inhibition of Rous sarcoma viral RNA translation by a specific oligodeoxynucleotide. Proc. Natl. Acad. Sci. USA 74, 4370-4373 (1977); Opalinska, J. B. & Gewirtz, A. M. Nucleic-acid therapeutics: basic principles and recent applications. Nature Rev. 1 (July), 1-10 (2002)]. Of these, the hybridization-driven “antisense” strategies, using ribozymes, DNAzymes, and antisense oligonucleotides such as chimeric RNA-DNA (gapmers) or phosphorothioate DNA have received the greatest attention and are the subject of numerous reviews [Stull, R. A. & Szoka, F. C. Antigene, ribozyme and aptamer nucleic acid drugs: progress and prospects. Pharmaceutical Res. 12, 465-483 (1995); Uhlmann E. and Peyman, A. Antisense oligonucleotides: a new therapeutic principle. Chem. Rev. 90, 544-584 (1990)]. More recently, post-transcription gene silencing or RNA interference (RNAi) has emerged as an exciting potential alternative to these more classical approaches [Elbashir, S. M, Lendeckel, W. & Tuschl, T. RNA interference is mediated by 21- and 22-nucleotide RNAs. Genes Dev. 15, 188-200 (2001); Caplen, N. J. et al. Specific inhibition of gene expression by small dsRNAs in invertebrate and vertebrate systems. Proc. Natl. Acad. Sci. USA 98, 9742-9747 (2001); Nishikura, K. A short primer on RNAi: RNA-directed RNA polymerase acts as a key catalyst. Cell 107, 415-418 (2001); Tuschl, T. Expanding small RNA interference. Nature Biotechnol. 20, 446-448 (2002); Mittal, V. Improving the efficiency of RNA interference in mammals. Nature Rev. 5, 355-365 (2004); Nykanen A., Haley, B. & Zamore, P. D. ATP requirements and small interfering RNA structure in the RNA interference pathway. Cell 107, 309-321 (2001)]. There are numerous reports describing the utility of this method for silencing genes in living organisms ranging from yeast to mammals [Yu, J. Y., S. L. DeRuiter, and D. L. Turner, RNA interference by expression of short-interfering RNAs and hairpin RNAs in mammalian cells. Proc. Natl. Acad. Sci. USA 99, 6047 (2002); Donze, O. and D. Picard, RNA interference in mammalian cells using siRNAs synthesized with T7 RNA polymerase. Nucleic Acids Res. 30, e46 (2002); Sui, G., C. Soohoo, B. Affar el, et al. A DNA vector-based RNAi technology to suppress gene expression in mammalian cells. Proc. Natl. Acad. Sci. USA 99, 5515 (2002); Paddison, P. J., A. A. Caudy, E. Bernstein, et al. Short hairpin RNAs (shRNAs) induce sequence-specific silencing in mammalian cells. Genes Dev. 16, 948 (2002)].

The utility of siRNA in vivo and its possible application in pharmacotherapy, as with other oligonucleotide-based therapies, faces some key hurdles (e.g., delivery, cellular uptake and biostability of oligonucleotides). There is a need to develop chemical modifications that result in clinically useful molecules. Initial work with antisense and siRNA oligonucleotides was undertaken with unmodified, natural molecules. It soon became clear however, that native oligonucleotides were subject to relatively rapid degradation, primarily through the action of 3′ exonucleases, but as a result of endonuclease attack as well. Oligoribonucleotides (RNA) are, in fact, generally more susceptible to nuclease degradation relative to DNA.

Antisense and siRNA molecules are now routinely modified to enhance their stability, as well as the strength of their hybridization with RNA since these physical attributes are often necessary for their therapeutic application [Mangos, M. M. & Damha, M. J. Flexible and frozen sugar-modified nucleic acids—modulation of biological activity through furanose ring dynamics in the antisense strand, Curr. Top. Med. Chem. 2, 1145-1169 (2002); Agrawal, S. and Q. Zhao. Mixed backbone oligonucleotides: improvement in oligonucleotide-induced toxicity in vivo. Antisense Nucleic Acid Drug Dev. 8, 135 (1998); Crooke, S. T. Molecular mechanisms of action of antisense drugs. Biochim. Biophys. Acta 1489, 31 (1999); Micklefield, J. Backbone modification of nucleic acids: synthesis, structure and therapeutic applications. Curr. Med. Chem. 8, 1157 (2001); Nielsen, P. E., Antisense peptide nucleic acids. Curr. Opin. Mol. Ther. 2, 282 (2000); Braasch, D. A., S. Jensen, Y. Liu, et al., RNA interference in mammalian cells by chemically-modified RNA. Biochemistry 42, 7967 (2003)]. In the presence of a delivery vehicle, both types of molecules are able to cross cell membranes and then to hybridize with their intended RNA target. RNA tertiary structure is an important factor governing the ability of antisense oligonucleotides [Opalinska, J. B., A. Kalota, L. K. Gifford, et al. Oxetane modified, conformationally constrained, antisense oligodeoxyribonucleotides function efficiently as gene silencing molecules [Nucleic Acids Res. 32, 5791 (2004). Scherr, M., J. J. Rossi, G. Sczakiel, et al., RNA accessibility prediction: a theoretical approach is consistent with experimental studies in cell extracts. Nucleic Acids Res. 28, 2455 (2000). Sokol, D. L., X. Zhang, P. Lu, et al., Real time detection of DNA.RNA hybridization in living cells. Proc. Natl. Acad. Sci. USA 95, 11538 (1998)] and siRNA [Opalinska, J. B., A. Kalota, L. K. Gifford, et al. Oxetane modified, conformationally constrained, antisense oligodeoxyribonucleotides function efficiently as gene silencing molecules. Nucleic Acids Res. 32, 5791 (2004); Scherr, M., J. J. Rossi, G. Sczakiel, et al., RNA accessibility prediction: a theoretical approach is consistent with experimental studies in cell extracts. Nucleic Acids Res. 28, 2455 (2000); Sokol, D. L., X. Zhang, P. Lu, et al., Real time detection of DNA.RNA hybridization in living cells. Proc. Natl. Acad. Sci. USA 95, 11538 (1998)] to hybridize with their target. It goes without saying that it is undesirable for either type of molecule to exert non-sequence specific binding. Meeting all these requirements has turned out to be a demanding task.

Unmodified siRNA duplexes have been used with success for gene silencing, however, chemical modification of one or both of the strands will likely be necessary for therapeutic applications in order to improve biostability and pharmacokinetic properties. Numerous chemical modifications have been tested for effects on siRNA activity, although it is not clear yet which of these modifications will be the most advantageous. In designing new analogues, it is important to recognize that two key features of siRNA differ from traditional antisense approaches: (i) duplex RNAs are recognized and (ii) gene inhibition involves RISC(RNA-Induced Silencing Complex)—rather than RNase H—to promote recognition and cleavage of the mRNA target [Elbashir, S. M, Lendeckel, W. & Tuschl, T. RNA interference is mediated by 21- and 22-nucleotide RNAs. Genes Dev. 15, 188-200 (2001); Caplen, N. J. et al. Specific inhibition of gene expression by small dsRNAs in invertebrate and vertebrate systems. Proc. Natl. Acad. Sci. USA 98, 9742-9747 (2001); Nishikura, K. A short primer on RNAi: RNA-directed RNA polymerase acts as a key catalyst. Cell 107, 415-418 (2001); Tuschl, T. Expanding small RNA interference. Nature Biotechnol. 20, 446-448 (2002); Mittal, V. Improving the efficiency of RNA interference in mammals. Nature Rev. 5, 355-365 (2004)]. As such, RNA-like oligonucleotides are prime candidates for introducing sugar or backbone modifications without perturbing the overall A-form helical structure they require for activity. A promising modification is Locked Nucleic Acids (LNA), in which key benefits were achieved with relatively few modifications that do not significantly compromise siRNA activity (e.g., improved thermal stability and biostability, and reduced off target-effects) [Elmen, J. et al. Locked nucleic acid (LNA) mediated improvements in siRNA stability and functionality. Nucl. Acids Res. 33, 439-447 (2005)]. However, the activity and specificity of such compounds was found to be highly dependent on the site and degree of the LNA modifications. A single LNA substitution at the 5′-end of the antisense strand abolished activity. Moreover, activity was significantly impaired when the antisense strand was modified, whereas sense strand LNA modifications were only tolerated with slightly modified oligonucleotides, displaying equal or lower activity than unmodified siRNA. There appears to be limitations with other chemistries, including toxicity (phosphorothioate-RNA) and impaired activity (2′F-RNA, boranophosphate-RNA), with increasing degrees of modification [Amarzguioui, M. et al. Tolerance for mutations and chemical modifications in a siRNA, Nucl. Acids. Res. 31, 589-595 (2003)]. While this may in principle be compensated by the nuclease stability and/or specificity imparted by certain oligonucleotide chemistries, the prediction of effective siRNA chemistries remains an active focus of continued studies.

There is a need for chemically modified siRNAs that have nuclease stability and/or the ability to inhibit gene expression.

### SUMMARY OF THE INVENTION

According to one broad aspect of the invention, a small interfering RNA (siRNA) for modulating expression of a target gene in a sequence-specific manner comprising a double stranded duplex wherein at least one ribonucleic acid nucleotide of the siRNA is substituted with an arabinose modified nucleotide is provided. The arabinose modified nucleotide is 2′-deoxy-2′-fluoroarabinonucleotide (FANA).

Preferably, the siRNA is 15-30 nucleotides in length and has 1-3 nucleotide overhangs at the 3′ and 5′ termini.

In specific embodiments, the duplex may have any number of arabinonucleotides at any location at either the sense or the antisense strand, for example:

### 5′-ARARARARARARARARARARA-3′

### 5′-AARRAARRAARRAARRAARRA-3′

### 5′-AARRRRRRRRRRRRRRRRRRR-3′

### 5′-RRRRRRRRRRRRRRRRRRRRRAA-3′

etc.
wherein A is an arabinonucleotide and R is a ribonucleotide.

In other embodiments of the invention, the sense strand is fully substituted with arabinonucleotides. For example:

5′-AAAAAAAAAAAAAAAAAAAAA-3′

and the antisense strand is an all-RNA strand or partially substituted RNA strand, for example:

5′-RRRRRRRRRRRRRRRRRRRRRRR-3′

5′-RRRRRRRRRRRRRRRRRRRRRAA-3′

5′-AARRRRRRRRRRRRRRRRRRRRR-3′

etc.

In other embodiments of the invention, the arabinonucleotide comprises a 2′ substituent selected from the group consisting of fluorine, hydroxyl, amino, azido, alkyl, alkoxy, and alkoxyalkyl groups. In a further embodiment of the invention, the alkyl group is selected from the group consisting of methyl, ethyl, propyl, butyl, and functionalized alkyl groups such as ethylamino, propylamino and butylamino groups. In embodiments, the alkoxy group is selected from the group consisting of methoxy, ethoxy, proproxy and functionalized alkoxy groups such as —O(CH₂)_{q}—R, where q=2-4 and —R is a —NH₂, —OCH₃, or —OCH₂CH₃ group. In embodiments, the alkoxyalkyl group is selected from the group consisting of methoxyethyl, and ethoxyethyl. In embodiments, the 2′ substituent is fluorine and the arabinonucleotide is a 2′-fluoroarabinonucleotide (FANA). Preferably, the FANA nucleotide is araF-G, araF-T, araF-U, araF-A, araF-5-methyl-C.

According to some embodiments of the invention, the siRNA is for decreasing any one of luciferase expression, CCR3 expression, or PDE4D expression.

According to another embodiment of the invention, the siRNA is for decreasing Respiratory Syncytial Virus replication.

In other embodiments of the invention, the duplex comprises one or more internucleotide linkages selected from the group consisting of:

a) phosphodiester;

b) phosphotriester;

c) phosphorothioate;

d) methylphosphonate;

e) boranophosphate and

f) any combination of (a) to (e).

According to another broad aspect of the invention, a method is provided for increasing at least one of nuclease stability and modulation of target gene activity of an siRNA comprising replacing at least one nucleotide of the siRNA with an arabinose modified nucleotide, preferably 2′-deoxy-2′-fluoroarabinonucleotide (FANA).

According to another broad aspect of the invention a pharmaceutical composition is provided, comprising the siRNA of the present invention along with a pharmaceutically acceptable carrier.

According to another broad aspect of the invention, use of the siRNA of the present invention is provided for the preparation of a medicament for modulating expression of a target gene, preferably one of CCR3 and PDE4D.

According to another embodiment of the invention, use of the siRNA of the present invention is provided for the preparation of a medicament for decreasing Respiratory Syncytial Virus replication.

According to another broad aspect of the invention, a method of modulating gene expression in a patient in need thereof is provided. The method comprises administering to the patient a therapeutically effective amount of the pharmaceutical composition of the invention. Preferably, the pharmaceutical composition comprises a siRNA for any one of decreasing expression of CCR3, decreasing expression of PDE4D, and decreasing Respiratory Syncytial Virus replication.

According to another broad aspect of the invention a commercial package is provided. The commercial package comprises the pharmaceutical composition of the present invention together with instructions for its use for modulating gene expression. Preferably, the pharmaceutical composition comprises an siRNA for any one of decreasing CCR3 expression, decreasing expression of PDE4D and decreasing Respiratory Syncytial Virus replication.

### BRIEF DESCRIPTIONS OF THE DRAWINGS

The invention will now be described in greater detail having regard to the appended drawings in which:

FIG. 1 illustrates the efficacy of the different siRNAs at inhibiting luciferase in HeLa X1/5 cells. Cells were transfected with 0.21 μg of siRNA having modifications in the sense strand only (A), in the antisense strand only (B) or in both sense and antisense strands (C). Luciferase activity levels were measured 24 h post-transfection and normalized to metabolic activity. The normalized luciferase activity was then determined as a percentage of luciferase activity as compared to a control siRNA set at 100%. Data represents mean normalized luciferase activity+/−SEM. Luciferase mRNA levels were quantified by real-time PCR analysis (relative to expression of the house keeping gene GAPDH) 24 h post-transfection. Bars show mean Luciferase/GAPDH ratios+/−SEM.

FIG. 2 shows the potency of FANA-containing siRNA at inhibiting the luciferase activity. Dose-responses were obtained for each siRNA by transfecting cells with different amounts of active siRNA for 24 h. Dose-responses for siRNA having modifications in the sense strand only are shown in (A), in the antisense strand only (B) or in both sense and antisense strands (C). Luciferase activity was measured and values normalized to the metabolic activity and compared to a control siRNA set at 100%. The data represent mean normalized luciferase activity+/−SEM.

FIG. 3 illustrates efficacy over time of different siRNA targeting the luciferase mRNA in HeLa X1/5 cells. Cells were transfected with 0.21 μg of siRNA. Luciferase activity was measured 4, 8, 24, 48, 72 and 96 h post-transfection. The data represent mean normalized luciferase activity+/−SEM compared to a control siRNA set at 100%.

FIG. 4 illustrates the serum stability of FANA-containing siRNA. The different siRNAs were incubated in 10% fetal bovine serum at 37° C. and aliquots were taken at the time points as indicated. The siRNAs were separated by PAGE and visualized with SYBR gold. Bands were quantified by densitometry and the percentage of intact siRNA from initial amount set at 100%. A) Serum stability of siRNAs targeting luciferase is shown. “ds” depicts double-stranded siRNA marker and “ss” single-stranded. B) Graph representing serum stability of different siRNAs targeting luciferase. C) Graph representing serum stability of different siRNAs targeting CCR3. D) Graph representing serum stability of different siRNAs targeting PDE4D. Data represent mean values from two to three independent experiments±SEM.

FIG. 5 illustrates the efficacy of FANA-containing siRNAs at inhibiting rat CCR3 expression in NIH-3T3 cells. Increasing amounts of siRNAs targeting the rat CCR3 were co-transfected with a plasmid expressing the rat CCR3 gene in NIH-3T3 cells. CCR3 mRNA expression levels were measured 24 h post-transfection using the Quantigene (Panomics) method and normalized to the expression levels of a reference gene (luciferase). The activity of the siRNA was determined as the percentage inhibition compared to a control siRNA set at 100%. Data represents mean+/−SEM (n=6).

FIG. 6 illustrates the efficacy of FANA-containing siRNAs targeting the RSV viral P-protein on RSV production in A549 cells. A549 cells were cultured and seeded at 0.1×10⁵ cells per well in 24-well plates and cultured overnight at 37° C., 5% CO₂. The following day, cell cultures were transfected with 0.05 ug, 0.2 ug, or 0.4 ug of siRSV-P2 (siRNA against RSV viral P-protein), siRSV-P2-Mi (siRNA mismatch against RSV viral P-protein) siRSV-P2-O/F4 and negative control siRNA-P2-Mi-O/F4 using Lipofectamine2000 transfection reagent at a ratio of siRNA:Lipofectamine 2000 of 1:3. Each transfection experiment was performed in triplicate. One day post-transfection, cells were infected with hRSV at a M.O.I=1 and incubated at 37° C., 5% CO₂ overnight. Supernatants were harvested 24 hrs post-infection and assessed by ELISA for viral levels by quantification of RSV protein. Data is expressed as % RSV inhibition by siRNA relative to levels of RSV inhibition by their respective mismatch siRNA.

### DETAILED DESCRIPTION

This invention relates to modified oligonucleotide duplexes designed to target mRNA and promote mRNA degradation via the RNAi mechanism. In particular, selective inhibition of luciferase activity, rat CCR3 expression and RSV viral replication using short interfering RNA duplexes containing modified arabinonucleotides (FANA) is shown. The methods of RNAi described herein are in contrast to the common methods described above, which have concentrated on the use of modified nucleotides derived from the naturally occurring units (i.e., DNA, RNA, 2′-OMe-RNA, 2′F-RNA nucleotides) [Allerson, C. R. et al. Fully 2′-modified oligonucleotide duplexes with improved in vitro potency and stability compared to unmodified small interfering RNA. J. Med. Chem. 48, 901-904 (2005)].

This invention encompasses the characterization of a series of sugar modified duplexes that inhibit gene expression in a human cell line. These small interfering duplexes contain arabinose modified nucleotides conferring improved characteristics on the duplex, such as improved stability against nucleases present in body fluid. Preferably, the sugar modified nucleotides are 2′-deoxy-2′-fluoroarabinonucleotides (FANA). The method for generating the FANA modified duplexes necessitates the substitution of RNA nucleotides for FANA residues.

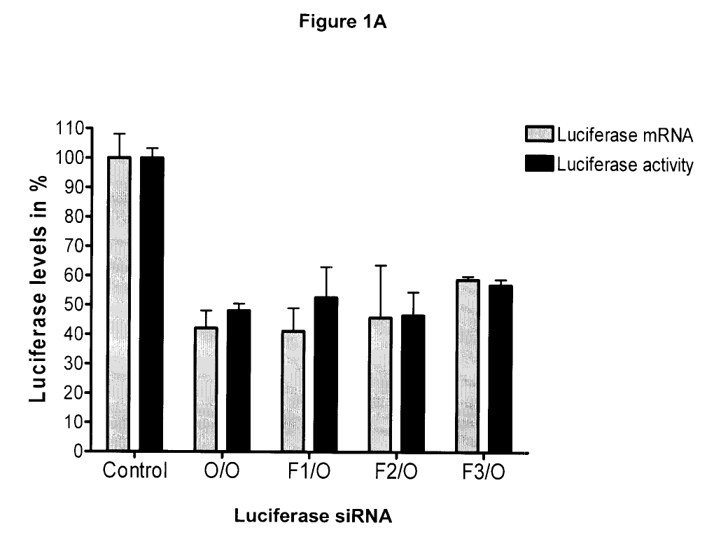

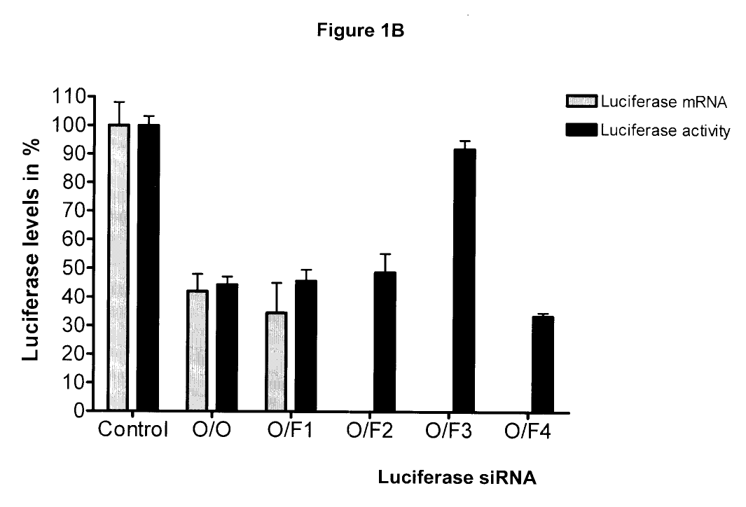

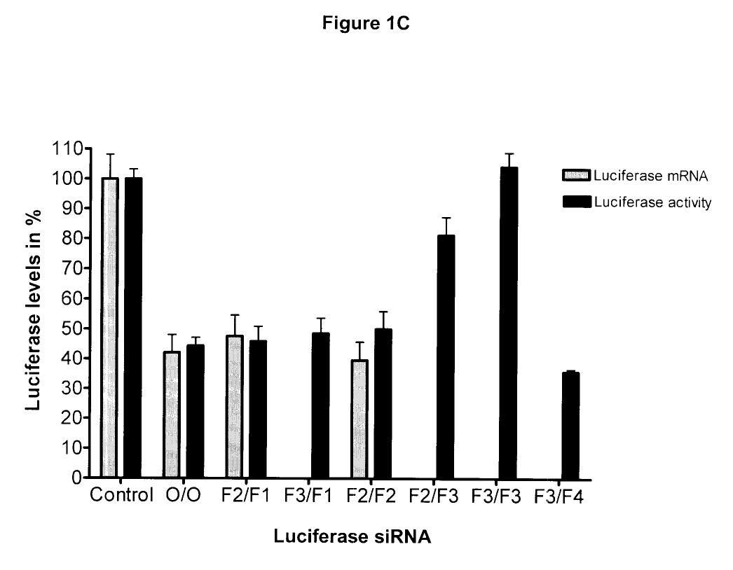

Activity of the modified siRNAs was evaluated using a modified HeLa cell line engineered to over-express luciferase. Luciferase mRNA expression levels and luciferase activity levels were determined using real-time PCR and luciferase assay techniques, respectively. Design and selection of the actual siRNA base sequence was performed according to Mittal et al. [Mittal, V. Improving the efficiency of RNA interference in mammals. Nature Rev. 5, 355-365 (2004)] utilizing the Ambion and Qiagen algorithms and NCBI Blast searches. At least three candidate siRNA duplexes were selected and tested as described above. Once the most active siRNA duplex was identified (EC₅₀ ˜0.5 nM), preliminary experiments were carried out to assess the impact of arabinose modification on siRNA activity. Selective, specific and efficient inhibition of luciferase activity of such FANA modified duplexes is demonstrated (FIG. 1). Complete replacement of one RNA strand (sense strand) in siRNA duplexes with a FANA strand generates FANA:RNA hybrids that also afford selective, specific and efficient downregulation of an mRNA target (FIG. 1A).

The compounds disclosed here represent the first examples of FANA modified duplexes (FANA modified siRNAs, and FANA:RNA hybrids) capable of inhibiting gene expression selectively via the RNAi mechanism.

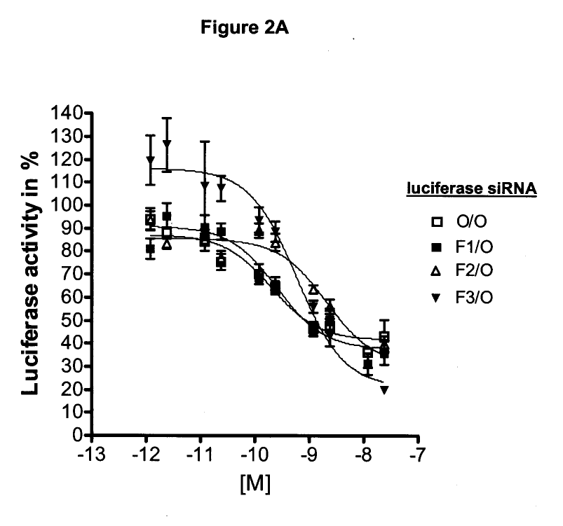

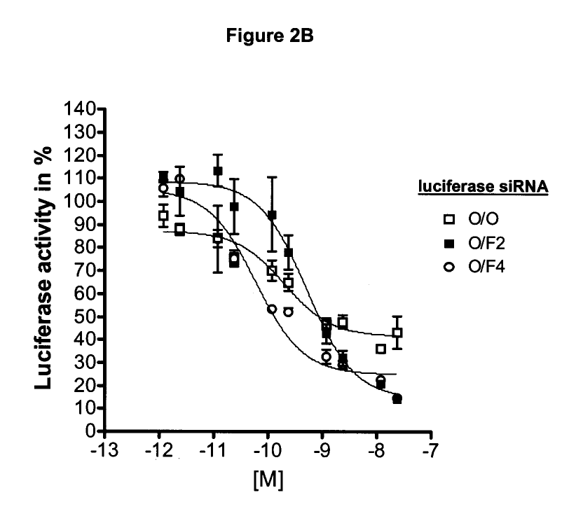

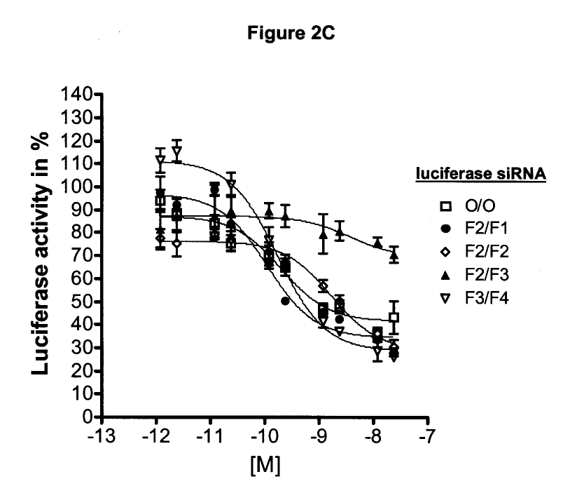

Specifically, this invention provides FANA nucleotides that are compatible with the activity of siRNA duplexes. In addition, it is shown that an entire FANA sense strand can bind to a complementary unmodified RNA antisense strand generating a duplex that enters the RNAi pathway to selectively and efficiently target a mRNA and promote its degradation (FIGS. 1A and 2A). These modified duplexes are obtained by synthesizing the constituent strands (via solid-phase chemical methods) and then allowing them to anneal to form a duplex. Unexpectedly, in all cases involving partial or full modification of the sense strand, gene silencing activity is similar to that observed with the unmodified native siRNA duplexes (FIGS. 1A and 2A). Treatment with FANA modified duplexes resulted in a reduction of luciferase activity in a concentration-dependent manner with an estimated EC₅₀ in the 0.06 to 3.6 nM range. The potencies observed for the sense modified duplexes were comparable to those of the native siRNA (FIGS. 2A-2C), which had an estimated EC₅₀ of 0.20 nM in this system (Table 1).

This invention also provides RNA duplexes in which an unmodified sense strand is annealed to an antisense strand in which the dangling dN terminal residues (3′ or 5′-termini) are replaced with FANAs without affecting activity (FIGS. 1B and 2B). Surprisingly, substituting the two 3′-deoxynucleotides with FANA residues confers increased potency over unmodified siRNA (FIGS. 1B and 2B), in striking contrast to siRNAs with LNA modifications, where the corresponding changes resulted in a significant decrease or complete loss of activity [Elmen, J. et al. Locked nucleic acid (LNA) mediated improvements in siRNA stability and functionality. Nucl. Acids Res. 33, 439-447 (2005)].

This invention also provides RNA duplexes in which both sense and antisense strand contain modified residues while maintaining RNAi activity (FIG. 1C). As for the RNA duplexes containing FANAs on one of the two strands, these duplexes showed specific target degradation at potencies equal to or greater than that of unmodified siRNA (FIG. 2C).

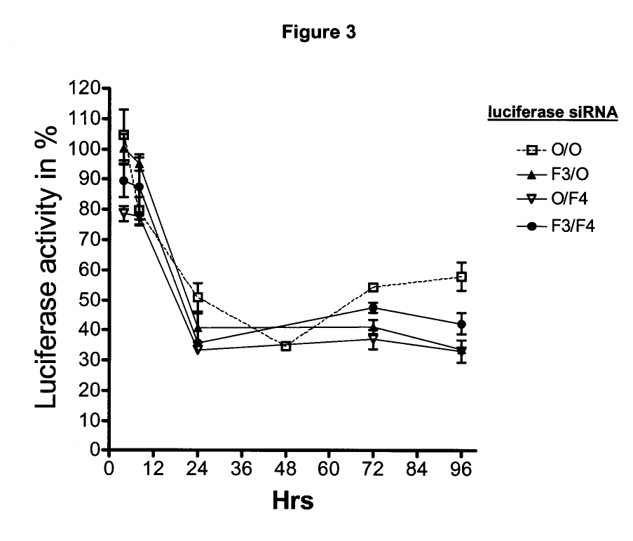

Similar to unmodified siRNAs, sustained inhibition of luciferase activity was observed when arabinose modified duplexes were transfected into cells for up to 4 days post transfection (FIG. 3). However, at this time point, modified siRNAs were found to have greater inhibitory activity than unmodified siRNA.

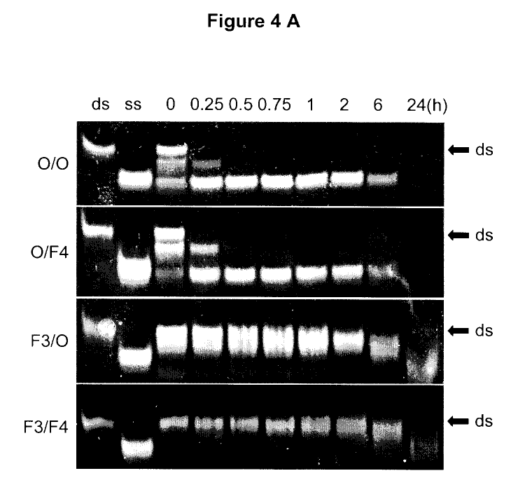

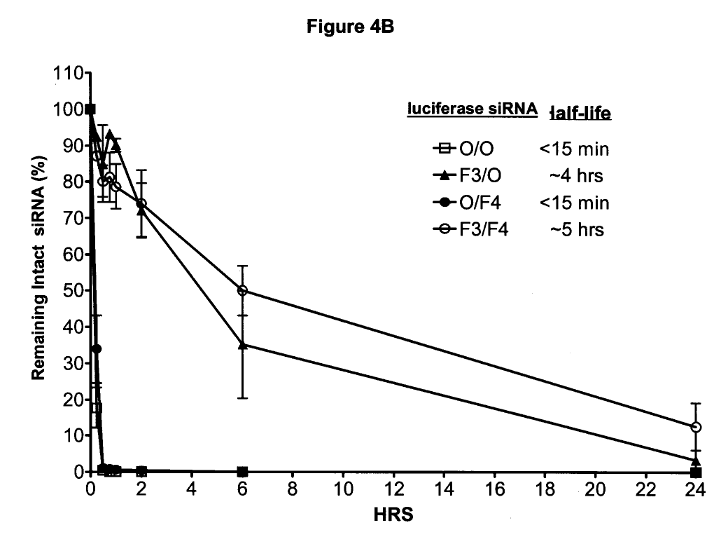

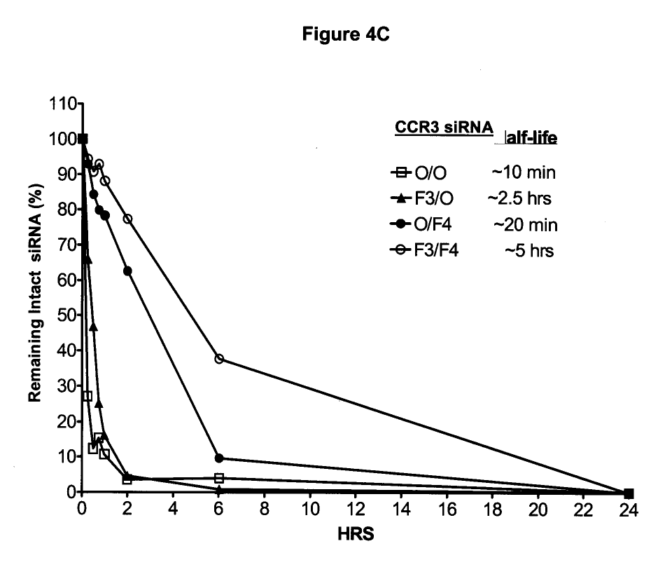

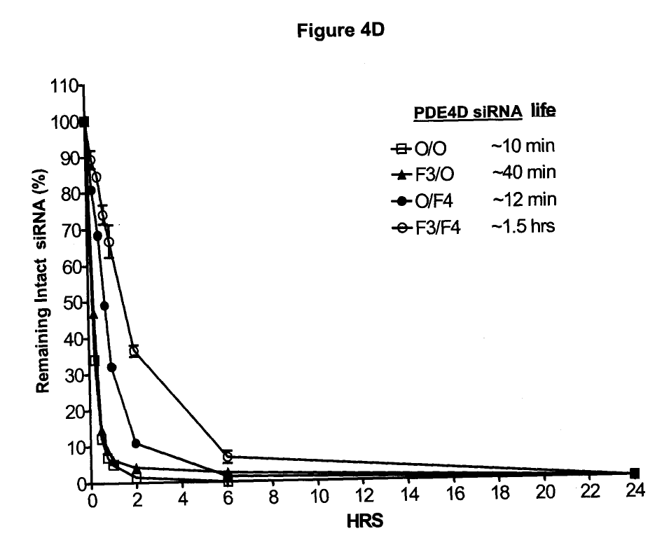

Herein is presented evidence that the nuclease stability of FANA containing siRNA duplexes is improved over unmodified siRNA duplexes (FIG. 4). Whereas unmodified siRNAs are completely degraded within 15 minutes, siRNA duplexes containing a fully modified sense strand and 3′-end overhang-modified antisense strand are readily detectable after 5 h. Accordingly, 2′-deoxy-2′-fluoro-β-D-arabino-(oligonucleotides), alone or in combination with ribonucleotide (RNA) units, are capable of hybridizing to complementary (antisense) RNA strands to generate siRNA duplexes with improved potency and increased nuclease resistance. These properties are highly desired in contemplating the in vivo administration of these compounds.

A “therapeutically effective amount” refers to an amount effective, at dosages and for periods of time necessary, to achieve the desired therapeutic result. A therapeutically effective amount of a modified nucleic acid of the invention may vary according to factors such as the disease state, age, sex, and weight of the individual, and the ability of the modified nucleic acid to elicit a desired response in the individual. Dosage regimens may be adjusted to provide the optimum therapeutic response. A therapeutically effective amount is also one in which any toxic or detrimental effects of the compound are outweighed by the therapeutically beneficial effects. For any particular subject, specific dosage regimens may be adjusted over time according to the individual need and the professional judgement of the person administering or supervising the administration of the compositions.

As used herein “pharmaceutically acceptable carrier” or “excipient” includes any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, an oligonucleotide duplex of the invention can be administered in a time release formulation, for example in a composition which includes a slow release polymer. The modified oligonucleotide can be prepared with carriers that will protect the modified oligonucleotide duplex against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating an active compound, such as an oligonucleotide duplex of the invention, in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. In accordance with an alternative aspect of the invention, an oligonucleotide duplex of the invention may be formulated with one or more additional compounds that enhance its solubility.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the claims, the word “comprising” is used as an open-ended term, substantially equivalent to the phrase “including, but not limited to”.

The following examples are illustrative of various aspects of the invention, and do not limit the broad aspects of the invention as disclosed herein.

### Example 1

### Chemical Synthesis of siRNA Duplexes and Arabinose Modified Duplexes

The sequence and composition of the oligomers prepared in this study are shown in Table 1. Syntheses of oligoribonucleotides, FANA modified oligoribonucleotides, as well as all-FANA oligonucleotides were carried out on a 1 μmol scale on an Applied Biosystems (ABI) synthesizer using the standard β-cyanoethylphosphoramidite chemistry according to published protocols [E. Viazovkina, M. M. Mangos, M. I. Elzagheid, and M. J. Damha (2002) Current Protocols in Nucleic Acid Chemistry, Unit 4.15); M. J. Damha and K. K. Ogilvie (1993) Oligoribonucleotide synthesis—the silyl-phosphoramidite method in “Protocols for Oligonucleotide and Analogs: Synthesis and Properties” S. Agrawal (ed.), Methods in Molecular Biology pp. 81-114, The Humana Press Inc., Totowa, N.J.]. FANA modified oligoribonucleotides and oligoribonucleotides were deprotected, purified and handled identically. All oligonucleotides were purified by anion exchange HPLC or gel electrophoresis, and desalted via size-exclusion chromatography using Sephadex G-25 beads. Stock solutions of duplexes were prepared by mixing the sense and corresponding antisense strands (1:1 stoichiometric ratio), lyophilizing the samples, and adding sufficient resuspension/annealing buffer to make a 20 μM solution. The composition of the siRNA resuspension/annealing buffer is 100 mm potassium acetate, 30 mM HEPES-KOH, 2 mM magnesium acetate, pH 7.4.

### Example 2

### Efficacy of FANA-Containing siRNA

This example relates to the efficacy of FANA-containing siRNAs with respect to the specific knockdown of the target mRNA and reduction of luciferase activity in HeLa X1/5 cells. The HeLa X1/5 cell line was obtained from ECACC (ECACC No. 95051229) and maintained in EMEM media supplemented (Invitrogen, Burlington ON) with 10% fetal bovine serum, 2 mM L-glutamine, 1% non-essential amino acids, 1% vitamins, 500 μg/ml G418 and 300 μg/ml Hygromycin. For transfection, 1.0×10⁵ cells/well were plated onto 24-well plates 24 hours prior to transfection. The day of transfection, cells were transfected with 0.21 μg of siRNA using Lipofectamine 2000 (Invitrogen, Burlington ON) at a siRNA:Lipofectamine 2000 ratio of 1:2 according to the manufacturers' recommendations. Cells were harvested 24 h post-transfection. Cell metabolic activity, as an indicator of cellular toxicity resulting from siRNA transfection, was assessed using the alamar Blue™ fluorimetric assay (Medicorp, Montreal QC) as per the manufacturers' recommendations.

Luciferase activity assays were performed using the luciferase assay system (BD Bioscience, Mississauga, ON) according to the manufacturer's protocol. Briefly, following exposure to the siRNA, cells were washed with phosphate-buffered saline (Invitrogen, Burlington ON) and lysed. Cell lysates were centrifuged to remove cellular debris and 20 μl aliquots were transferred to 96-well lumitrac plates (Ultident; Greiner Bio-one). Luminescence was measured using a microplate luminometer (Luminoskan Ascent, Thermo LabSystem) immediately following addition of the luciferin substrate solution. Luminescence values were then normalized to the cell metabolic activity values (alamar Blue™) to compensate for toxicity resulting from transfection.

For real-time PCR analysis, total RNA was extracted using the RNeasy mini kit (Qiagen, Mississauga ON) according to the manufacturers' protocol. cDNA was prepared from 1 μg total RNA using the SuperScrip™ II Reverse Transcriptase and random primers (Invitrogen, Burlington ON). Quantitative real-time PCR was performed using gene-specific primers and probes for the luciferase gene (LUC5013 F1: 5′-acgctgggcgttaatcagag-3′; LUC5013 R1: 5′-gtcgaagatgttggggtgttg-3′; TIB MOLBIOL) and the housekeeping gene GAPDH (huGAPD for: 5′-ggtggtctcctctgacttc-3′; huGAPD rev: 5′-ctcttcctcttgtgctcttg-3′; TIB MOLBIOL) using previously optimized conditions and the LightCycler (Roche, Laval QC).

Results presented in FIG. 1 indicate that FANA is well tolerated when incorporated into siRNA. Indeed, an siRNA having an all-FANA modified sense strand (F3/O) retained its activity (mRNA and luciferase activity) when compared to the unmodified siRNA (FIG. 1A). Our data also indicate that FANA modifications are well tolerated when introduced into the antisense strand (FIG. 1B). Replacement of the two 3′-overhang deoxynucleotides with two FANA residues (O/F4 and F3/F4) resulted in increased inhibitory activity (65%) of the duplex when compared to an unmodified siRNA duplex (55%) (FIGS. 1B and 1C). These results are in contrast to published data in which chemical modifications were shown to be well tolerated at one end only, depending on the type of modification. However, introduction of six FANAs, encompassing the 5′ end and the middle part of the antisense strand collectively, abolished activity of the siRNA duplex regardless of the modifications introduced onto the sense strand (FIGS. 1B and 1C). It is known that the middle portion of the antisense strand of the siRNA is important for duplex interaction with the RNAi cellular machinery and is very sensitive to chemical modifications. Preferably, therefore, modifications on both the sense and antisense strands are within the parameters as described above (FIG. 1C).

### Example 3

### Potency of FANA-Containing siRNA

This example relates to the potency of FANA-containing siRNAs with respect to the specific knockdown of luciferase activity in HeLa X1/5 cells. Dose-response studies were performed using a total amount of siRNA of 0.21 μg whereby the effective siRNA was serially diluted with a control siRNA, reducing the effective amount of active oligonucleotide while keeping the final amount of siRNA constant. Cells were harvested 24 h post-transfection and luciferase activity determined.

Results indicate that an siRNA having two deoxynucleotides of the 3′-overhang of the antisense strand replaced with FANAs and having an unmodified (O/F4) or fully modified (F3/F4) sense strand inhibits luciferase activity in a concentration-dependent manner with increased potency over the counterpart unmodified siRNA (FIGS. 2B and 2C). The estimated EC50 values are presented in Table 1.

### Example 4

### Duration of Action of FANA-Containing siRNA

This example shows that FANA-containing siRNAs have sustained inhibitory activity up to 96 h. Luciferase activity was measured at different time points following exposure to the different modified and unmodified siRNAs (FIG. 3). Results indicate that siRNAs containing FANA residues have prolonged activity for up to 4 days. Moreover, the data demonstrate that FANA-containing siRNA have increased inhibitory activity at the 96 h time point when compared to the unmodified siRNA (65% vs. 45% inhibition of the luciferase activity, respectively).

### Example 5

### Increased Stability of FANA-Containing siRNA

This example relates to siRNA duplex stability in the presence of fetal bovine serum. Results of experiments are presented in FIG. 4. siRNAs were diluted in 10% fetal bovine serum in DMEM and incubated at 37° C. Aliquots of 12 μl were collected after 0.25, 0.5, 0.45, 1, 2, 6 and 24 h and frozen in 36 μl of 1.5×TBE-loading buffer containing 50% Ficoll until analysis. Samples were separated on 20% polyacrylamide gels under non-denaturing conditions and stained with SYBR gold (Invitrogen, Burlington ON). Bands corresponding to intact siRNA were quantified by densitometry analysis.

Results show that incorporation of FANAs in the sense strand confers significant resistance to serum-mediated siRNA degradation. Introduction of FANAs significantly enhances serum resistance of siRNAs. A representative gel is shown in FIG. 4A. All the unmodified forms of siRNA (O/O), regardless of the sequence, have half-lives shorter than 15 minutes (FIGS. 4B, 4C and 4D). Substitution of the two 3′-overhang deoxynucleotides in the antisense strand with two FANAs (O/F4) had no impact on the serum stability properties of the siRNA duplexes (FIGS. 4B, 4C and 4D). However, having an all FANA-modified sense strand (F3/O) conferred significant resistance to serum-mediated degradation as siRNA duplex half-lives of up to 4 h were observed (FIG. 4B). Finally, FANA modification of both the sense and antisense strands (F3/F4) resulted in even greater resistance to nuclease as siRNA half-lives of approximately 5 h were determined (FIGS. 4B and 4C).

### Example 6

### Efficacy of FANA-Containing siRNA at Inhibiting CCR3 mRNA Expression Levels

This example relates to the efficacy of FANA-containing siRNAs in specific knockdown of the expression levels of CCR3 mRNA in NIH-3T3 cells. The NIH-3T3 cell line was obtained from ATCC (ATCC CRL-1658) and maintained in DMEM medium (Invitrogen, Burlington ON) supplemented with 10% calf bovine serum, 4 mM L-glutamine, 3.7 g/L sodium bicarbonate, 4.5 g/l glucose and 1% Penicillin/Streptomycin. 1.0×10⁵ cells/well were seeded onto 24-well plates one day prior to transfection. Cells were transfected with 0.2 μg of plasmid expressing the rat CCR3 gene, 0.2 μg of plasmid expressing luciferase (reference gene) and 0.01, 0.1 or 0.2 μg of siRNA using Lipofectamine 2000 (Invitrogen, Burlington ON) at a DNA/siRNA:Lipofectamine 2000 ratio of 1:2 according to the manufacturers' recommendations. Cells were harvested 24 h following transfection. Expression levels of CCR3 and luciferase were quantified using the Quantigene method (Panomics, Fremont Calif.). CCR3 expression levels were then normalized to the levels measured for luciferase.

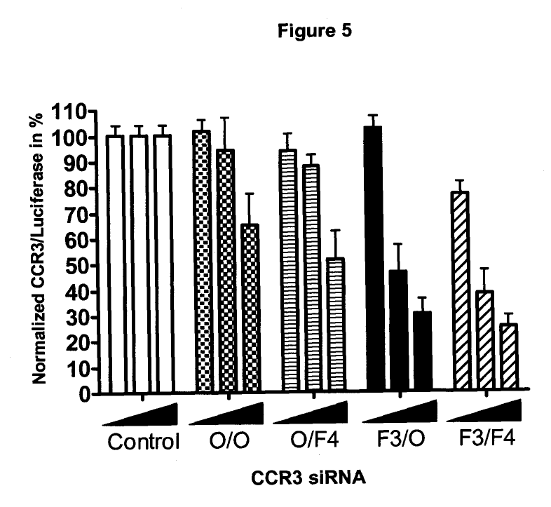

Results presented in FIG. 5 indicate that incorporation of FANA residues into siRNA resulted in a dose-dependent increase in the inhibitory activity of an siRNA targeting the rat CCR3 mRNA. Indeed, substitution of the two 3′-overhang deoxynucleotides in the antisense strand with two FANAs (O/F4) resulted in increased inhibitory activity of the duplex (up to 49% when compared to an unmodified CCR3 siRNA (35%)) (FIG. 5). In addition, a CCR3 siRNA having an all-FANA modified sense strand (F3/O) was more active (75% inhibition of CCR3 mRNA levels) when compared to the unmodified siRNA (FIG. 5). Finally, modification of both strands of the siRNA duplex was well tolerated with inhibitory activity reaching 75%. These data support the observation that FANA is: 1) well tolerated when introduced in siRNA duplexes and 2) enhances the inhibitory activity of the siRNA duplex.

### Example 7

### Increased Efficacy of FANA-Containing siRNA at Inhibiting Viral Replication

This example relates to the efficacy of siRNA duplexes containing FANA residues to inhibit replication of respiratory syncytial virus (RSV) in A549 cells. The A549 cell line (ATCC, # CCL-185) was maintained in Ham F12 medium (HyClone, Logan Utah) supplemented with 10% non-inactivated FBS (HyClone). 1.0×10⁵ cells were seeded into individual wells of 24-well plates one day prior to transfection. On the day of transfection, cells were transfected with 0.05 μg, 0.2 μg or 0.4 μg of siRNA at a 1:3 ratio of siRNA to transfection reagent (Lipofectamine 2000 (Invitrogen, Burlington ON)) according to the manufacturers' recommendations. 24 hours post-transfection cells were infected with RSV at a multiplicity of infection (M.O.I.) of 1 and the viral infection was allowed to proceed for one day. 24 hours after exposure to virus, cell supernatants were harvested and RSV levels were assessed using an ELISA method to detect RSV proteins.

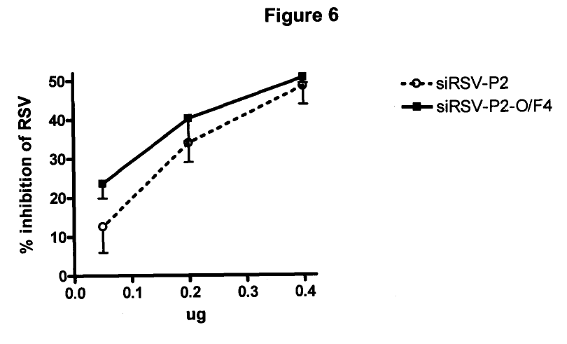

Results indicate that an siRNA duplex, wherein the two deoxynucleotides of the 3′ overhang of the antisense strand are substituted with FANAs and the sense strand remains unmodified (O/F4), inhibits RSV replication in a concentration-dependent manner having increased inhibitory activity compared to unmodified siRNA at lower doses (FIG. 6). These results support the observation that FANA increases the inhibitory activity of siRNAs.

All references cited are incorporated by reference herein. Although preferred embodiments of the invention have been described herein, it will be understood by those skilled in the art that variations may be made thereto without departing from the spirit of the invention or the scope of the appended claims.

TABLE 1
Oligonucleotides and duplexes synthesized in this study

| | | | | |
|---|---|---|---|---|
| | SEQ | | | EC₅₀ |
| Target | ID No. | Type | Sequence^{a} | (nM) |

| | | | | |
|---|---|---|---|---|
| Luciferase | | | | |
| O/O | 1 | RNA | 5′-GCUUGAAGUCUUUAAUUAAgg-3′ | 0, 20 |
| | 2 | | 3′-ggCGAACUUCAGAAAUUAAUU-5′ | |
| F1/O | 3 | RNA/FANA | 5′-GCUUGAAGUCUUUAAUUAATT-3′ | 0, 25 |
| | 4 | | 3′-ggCGAACUUCAGAAAUUAAUU-5′ | |
| F2/O | 5 | RNA/FANA | 5′-GCTTGAAGUCTTUAATTAAtt-3′ | 2, 0 |
| | 6 | | 3′-ggCGAACUUCAGAAAUUAAUU-5′ | |
| F3/O | 7 | RNA/FANA | 5′-GCTTGAAGTCTTTAATTAAGG-3′ | 0, 59 |
| | 8 | | 3′-ggCGAACUUCAGAAAUUAAUU-5′ | |
| O/F1 | 9 | RNA/FANA | 5′-GCUUGAAGUCUUUAAUUAAgg-3′ | 0, 16 |
| | 10 | | 3′-gGCGAACUUCAGAAAUUAAUU-5′ | |
| O/P2 | 11 | RNA/FANA | 5′-GCUUGAAGUCUUUAAUUAAgg-3′ | 0, 52 |
| | 12 | | 3′-ggCGAACUUCAGAAAUUAAUT-5′ | |
| O/P3 | 13 | RNA/FANA | 5′-GCUUGAAGUCUUUAAUUAAgg-3′ | 6, 3 |
| | 14 | | 3′-ggCGAACTTCAGAAATTAATT-5′ | |
| O/F4 | 15 | RNA/FANA | 5′-GCUUGAAGUCUUUAAUUAAgg-3′ | 0, 06 |
| | 16 | | 3′-GGCGAACUUCAGAAAUUAAUU-5′ | |
| P2/F1 | 17 | RNA/FANA | 5′-GCTTGAAGUCTTUAATTAAtt-3′ | 0,11 |
| | 18 | | 3′-gGCGAACUUCAGAAAUUAAUU-5′ | |
| F2/F2 | 19 | RNA/FANA | 5′-GCTTGAAGUCTTUAATTAAtt-3′ | 1, 7 |
| | 20 | | 3′-ggCGAACUUCAGAAAUUAAUT-5′ | |
| F2/F3 | 21 | RNA/FANA | 5′-GCTTGAAGUCTTUAATTAAtt-3′ | 3, 6 |
| | 22 | | 3′-ggCGAACTTCAGAAATTAATT-5′ | |
| F2/F4 | 23 | RNA/FANA | 5′-GCTTGAAGUCTTUAATTAAtt-3′ | 0, 06 |
| | 24 | | 3′-GGCGAACUUCAGAAAUUAAUU-5′ | |
| F3/F1 | 25 | RNA/FANA | 5′-GCTTGAAGTCTTTAATTAAGG-3′ | 0, 24 |
| | 26 | | 3′-gGCGAACUUCAGAAAUUAAUU-5′ | |
| F3/F3 | 27 | RNA/FANA | 5′-GCTTGAAGTCTTTAATTAAGG-3′ | >10 |
| | 28 | | 3′-ggCGAACTTCAGAAATTAATT-5′ | |
| F3/F4 | 29 | RNA/FANA | 5′-GCTTGAAGTCTTTAATTAAGG-3′ | 0, 17 |
| | 30 | | 3′-GGCGAACUUCAGAAAUUAAUU-5′ | |
| Rat CCR3 | | | | |
| O/O | 31 | RNA | 5′-ACACCCUAUGAAUAUGAGUtt-3′ | n.d. |
| | 32 | | 3′-ttUGUGGGAUACUUAUACUCA-5′ | |
| O/F4 | 33 | RNA/FANA | 5′-ACACCCUAUGAAUAUGAGUtt-3′ | n.d. |
| | 34 | | 3′- TTUGUGGGAUACUUAUACUCA- 5′ | |
| P3/0 | 35 | RNA/FANA | 5′-ACACCCTATGP˜ATATGAGTTT-3′ | n.d. |
| | 36 | | 3′- ttUGUGGGAUACUUAUACUCA- 5′ | |
| F3/F4 | 37 | RNA/FANA | 5′-ACACCCTATGAATATGAGTTT-3′ | n.d. |
| | 38 | | 3′- TTUGUGGGAUACUUAUACUCA- 5′ | |
| Human | | | | |
| PDE4D | | | | |
| O/O | 39 | RNA | 5′-AAGAACUUGCCUUGAUGUAca-3′ | n.d. |
| | 40 | | 3′-ttUUCUUGAACGGAACUACAU-5′ | |
| O/F4 | 41 | RNA/FANA | 5′-AAGAACUUGCCUUGAUGUAca-3′ | n.d. |
| | 42 | | 3′-TTUUCUUGAACGGAACUACAU-5′ | |
| P3/O | 43 | RNA/FANA | 5′-AAGAACTTGCCTTGATGTACA-3′ | n.d. |
| | 44 | | 3′-ttUUCUUGAACGGAACUACAU-5′ | |
| F3/F4 | 45 | RNA/FANA | 5′-AAGAACTTGCCTTGATGTACA-3′ | n.d. |
| | 46 | | 3′-TTUUCUUGAACGGAACUACAU-5′ | |
| RSVP2 | | | | |
| O/O | 47 | RNA | 5′-CCCUACACCAAGUGAUAAUtt-3′ | n.d. |
| | 48 | | 3′-ttGGGAUGUGGUUCACUAUUA-5′ | |
| O/F4 | 49 | RNA/FANA | 5′-CCCUACACCAAGUGAUAAUtt-3′ | n.d. |
| | 50 | | 3′-TTGGGAUGUGGUUCACUAUUA-5′ | |

^{a}uppercase letters = RNA; lowercase letters = DNA; bold uppercase letters = FANA
e.g. O/O represents the unmodified siRNA while O/F1 represents an siRNA with an unmodified sense strand and the F1 modification in the antisense strand. n.d. = not determined.

1. A small interfering RNA (siRNA) for modulating expression of a target gene in a sequence-specific manner comprising a double-stranded duplex wherein at least one ribonucleic acid nucleotide of the siRNA is substituted with an arabinose modified nucleotide.

2. The siRNA of claim 1, being 15-30 nucleotides in length.

3. The siRNA of claim 2, having 1-3 nucleotide overhangs at the 3′ and 5′ termini.

4. The siRNA of claim 3, wherein the arabinose modified nucleotide has a 2′ substituent selected from the group consisting of fluorine, hydroxyl, amino, azido, alkyl, alkoxy, and alkoxyalkyl groups.

5. The siRNA of claim 4, wherein the alkyl group is selected from the group consisting of methyl, ethyl, propyl, butyl, and functionalized alkyl groups, the alkoxy group is selected from the group consisting of methoxy, ethoxy, proproxy and functionalized alkoxy groups and the alkoxyalkyl group is selected from the group consisting of methoxyethyl, and ethoxyethyl.

6. The siRNA of claim 5, wherein the functionalized alkyl group is selected from the group consisting of ethylamino, propylamino and butylamino group and the functionalized alkoxy group is selected from the group consisting of —O(CH₂)_{q}—R, where q=2-4 and —R is a —NH₂, —OCH₃, or —OCH₂CH₃ group.

7. The siRNA of claim 3, wherein the at least one arabinose modified nucleotide is 2′-deoxy-2′-fluoroarabinonucleotide (FANA).

8. The siRNA of claim 7, wherein the at least one arabinose modified nucleotide is on a sense strand of the siRNA.

9. The siRNA of claim 7, wherein the at least one arabinose modified nucleotide is on an antisense strand of the siRNA.

10. The siRNA of claim 8, wherein all nucleotides on the sense strand are substituted with FANA.

11. The siRNA of claim 7, wherein the at least one arabinose modified nucleotide is a dangling nucleotide of at least one of the overhangs.

12. The siRNA of claim 1 for decreasing luciferase expression.

13. The siRNA of claim 12 with the strands of the duplex having the base sequences of SEQ ID NOS. 1 and 2.

14. The siRNA of claim 1 with the strands of the duplex having the sequences selected from the group consisting of SEQ ID NOS. 3 and 4; SEQ ID NOS. 5 and 6; SEQ ID NOS. 7 and 8; SEQ ID NOS. 9 and 10; SEQ ID NOS. 11 and 12; SEQ ID NOS. 13 and 14; SEQ ID NOS. 15 and 16; SEQ ID NOS. 17 and 18; SEQ ID NOS. 19 and 20; SEQ ID NOS. 21 and 22; SEQ ID NOS. 23 and 24; SEQ ID NOS. 25 and 26; SEQ ID NOS. 27 and 28; and SEQ ID NOS. 29 and 30.

15. The siRNA of claim 1 for decreasing CCR3 expression.

16. The siRNA of claim 15 with the strands of the duplex having the base sequences of SEQ ID NOS. 31 and 32.

17. The siRNA of claim 1 with the strands of the duplex having the sequences selected from the group consisting of SEQ ID NOS. 33 and 34; SEQ ID NOS. 35 and 36; or SEQ ID NOS. 37 and 38.

18. The siRNA of claim 1 for decreasing Respiratory Syncytial Virus (RSV) viral replication.

19. The siRNA of claim 18 with the strands of the duplex having the base sequences of SEQ ID NOS. 47 and 48.

20. The siRNA of claim 1 with the strands of the duplex having the sequences consisting of SEQ ID NOS. 49 and 50.

21. The siRNA of claim 1 for decreasing PDE4D expression.

22. The siRNA of claim 21 with the strands of the duplex having the base sequences of SEQ ID NOS. 39 and 40.

23. The siRNA of claim 1 with the strands of the duplex having the sequences selected from the group consisting of SEQ ID NOS. 41 and 42; SEQ ID NOS. 43 and 44; and SEQ ID NOS. 45 and 46.

24. The siRNA of claim 1, containing at least one internucleotide linkage selected from the group consisting of phosphodiester, phosphotriester, phosphorothioate, methylphosphonate, boranophosphate and any combination thereof.

25. A method for increasing at least one of nuclease stability and target gene inhibition activity of an siRNA comprising replacing at least one nucleotide of the siRNA with an arabinose modified nucleotide.

26. The method of claim 25, wherein the arabinose modified nucleotide is 2′-deoxy-2′-fluoroarabinonucleotide (FANA).

27. The method of claim 25, wherein the siRNA is a double-stranded duplex and 15-30 nucleotides in length with 1-3 nucleotide overhangs at the 3′ and 5′ termini.

28. The method of claim 27, wherein the at least one nucleotide being replaced is on a sense strand of the siRNA.

29. The method of claim 27, wherein the at least one nucleotide being replaced is on an antisense strand of the siRNA.

30. The method of claim 28, wherein all nucleotides on the sense strand are replaced.

31. The method of claim 27, wherein the at least one nucleotide being replaced is on the overhang.

32. The method of claim 25, wherein the strands of the duplex have the base sequences of SEQ ID Nos. 1 and 2 and the siRNA double-stranded duplex decreases luciferase expression.

33. The method of claim 25, wherein the strands of the duplex have the sequences of SEQ ID Nos. 31 and 32 and the siRNA decreases CCR3 expression.

34. The method of claim 25, wherein the strands of the duplex have the sequences of SEQ ID Nos. 39 and 40 and the siRNA decreases PDE4D expression.

35. The method of claim 25, wherein the strands of the duplex have the sequences of SEQ ID Nos. 47 and 48 and the siRNA decreases Respiratory Syncytial Virus (RSV) replication.

36. A pharmaceutical composition comprising the siRNA of claim 1 along with a pharmaceutically acceptable carrier.

37. (canceled)

38. (canceled)

39. (canceled)

40. (canceled)

41. (canceled)

42. A method of modulating expression of a target gene in a patient in need thereof, comprising administering a therapeutically effective amount of the pharmaceutical composition of claim 36.

43. The method of claim 42 wherein the target gene is CCR3 and the pharmaceutical composition comprises the siRNA of claim 15.

44. The method of claim 42 wherein the target gene is a gene for Respiratory Syncytial Virus (RSV) replication and the pharmaceutical composition comprises the siRNA of claim 18.

45. The method of claim 42 wherein the target gene is PDE4D and the pharmaceutical composition comprises the siRNA of claim 21.

46. A commercial package comprising the composition of claim 36 together with instructions for its use for modulating expression of a target gene.

47. The commercial package of claim 46 wherein the target gene is CCR3 and the pharmaceutical composition comprises the siRNA of claim 15.

48. The commercial package of claim 46 wherein the target gene is a gene for Respiratory Syncytial Virus (RSV) replication and the pharmaceutical composition comprises the siRNA of claim 18.

49. The commercial package of claim 46 wherein the target gene is PDE4D and the pharmaceutical composition comprises the siRNA of claim 21.
